(19) United States
(12) Patent Application Publication
(10) Pub. No.: US 2023/0390120 A1
(43) Pub. Date: Dec. 7, 2023

(54) EXTREMITY MEDICAL DEVICE WATERPROOF COVER

(71) Applicants: Terrah Osborne-Barbera, Trabuco Canyon, CA (US); Garrett Barbera, Trabuco Canyon, CA (US)

(72) Inventors: Terrah Osborne-Barbera, Trabuco Canyon, CA (US); Garrett Barbera, Trabuco Canyon, CA (US)

(21) Appl. No.: 17/829,503

(22) Filed: Jun. 1, 2022

(51) Int. Cl. A61F 13/04

(52) U.S. Cl. CPC A61F 13/045; A61F 13/043

(57) ABSTRACT

The extremity medical device waterproof cover disclosed herein may comprise a waterproof extremity cover combined with digit covers and a plurality of traction pads to provide a user with a waterproof environment for their medical device and additional grip for their hands or feet. The extremity medical device waterproof cover may comprise any appropriate flexible, resilient, and waterproof material and may attach securely to a user's arm or leg by an appropriate cuff. The device is designed to overcome the shortcomings in the prior art, which specifically include the lack of ability to grip objects with the hand while wearing a cast cover or reducing the risk of slipping and falling while wearing such a cover on the foot.

### TECHNICAL FIELD OF THE INVENTION

The present invention relates in general to arm and leg covers, and, more specifically, to an extremity medical device waterproof cover.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

An extremity medical device may be any device installed onto or implanted within a human body extremity. By way of example, such devices may include arm or leg casts, intravenous lines, biometric monitors, insulin pumps, and the like installed on the arm or leg of a person. Such devices are well known in the prior art and are used on very large numbers of the population, both for temporary purposes and for long-term or nearly life-long needs.

These medical devices are often designed for environmental resistance so that they are less susceptible to contamination or infection, though often such devices may require an additional layer of protection in certain circumstances. An arm or leg cast, for example, which may be worn on a person for only a few months, must be kept both clean and dry to prevent the break down of the plaster and bandage materials. A person wearing such a cast must, thus, take extra precautions when cleaning, washing, bathing, or showering so as to prevent such a break down.

There are a number of solutions known in the prior art to address such an issue. Known generally as “cast covers”, the most common solution involves the use of a waterproof sleeve that is pulled over the arm or the leg, and over the medical device, with a sealing end to prevent water entry into the cover and the medical device within. Such covers may enclose just a hand and wrist combination, a hand and lower arm combination, a hand and entire arm combination, a foot and ankle combination, a foot and lower leg combination, or a foot and entire leg combination, as needed or desired.

Cast covers designed for use on an upper extremity tend to comprise a sleeve and a hand cover, with some embodiments further comprising individual digit covers into which a person's fingers may be inserted. The advantage of such digit covers is an ability for the person to move their fingers separately from one another, but the necessarily slippery surface of the waterproof material still prevents the person from holding on to slippery materials. By way of example, a person wearing such a cast cover while showering is not able to grasp or manipulate a bar of soap due to the slippery nature of the cover and the soap and the wet environment.

Cast covers designed for use on a lower extremity tend to comprise a sleeve and foot cover, with no known embodiments further comprising individual digit covers into which a person's toes may be inserted. This type of cast cover is only advantageous in covering a medical device on a person's lower extremity, but the necessarily slippery waterproof material presents a number of potential risks and challenges to the person. By way of example, a person wearing such a cast cover while showering is at a very high risk of slipping and falling due to the slippery nature of the cover and the wet environment.

Thus, there is a need in the art for an extremity medical device waterproof cover that may comprise a waterproof extremity cover combined with digit covers and a plurality of traction pads to provide a user with a waterproof environment for their medical device and additional grip for their hands or feet. The extremity medical device waterproof cover may comprise any appropriate flexible, resilient, and waterproof material and may attach securely to a user's arm or leg by an appropriate cuff. The device is designed to overcome the shortcomings in the prior art, which specifically include the lack of ability to grip objects with the hand while wearing a cast cover or reducing the risk of slipping and falling while wearing such a cover on the foot.

### BRIEF SUMMARY OF THE INVENTION

To minimize the limitations in the prior art, and to minimize other limitations that will be apparent upon reading and understanding the present specification, the present invention describes an extremity medical device waterproof cover.

It is an objective of the present invention to provide an extremity medical device waterproof cover that may comprise a main body.

It is another objective of the present invention to provide an extremity medical device waterproof cover that may comprise an arm sleeve.

It is another objective of the present invention to provide an extremity medical device waterproof cover that may comprise a hand cover.

It is another objective of the present invention to provide an extremity medical device waterproof cover that may comprise a plurality of finger covers.

It is another objective of the present invention to provide an extremity medical device waterproof cover that may comprise an arm cuff.

It is another objective of the present invention to provide an extremity medical device waterproof cover that may comprise a plurality of finger pads.

It is another objective of the present invention to provide an extremity medical device waterproof cover that may comprise a palm pad.

It is another objective of the present invention to provide an extremity medical device waterproof cover that may comprise an arm closure mechanism.

It is another objective of the present invention to provide an extremity medical device waterproof cover that may comprise a leg sleeve.

It is another objective of the present invention to provide an extremity medical device waterproof cover that may comprise a foot cover.

It is another objective of the present invention to provide an extremity medical device waterproof cover that may comprise a plurality of toe covers.

It is another objective of the present invention to provide an extremity medical device waterproof cover that may comprise a leg cuff.

It is another objective of the present invention to provide an extremity medical device waterproof cover that may comprise a plurality of toe pads.

It is another objective of the present invention to provide an extremity medical device waterproof cover that may comprise a heel pad.

It is another objective of the present invention to provide an extremity medical device waterproof cover that may comprise a leg closure mechanism.

It is another objective of the present invention to provide an extremity medical device waterproof cover that may comprise a resilient material of construction.

It is another objective of the present invention to provide an extremity medical device waterproof cover that may comprise a water-proof material of construction.

It is another objective of the present invention to provide an extremity medical device waterproof cover that may comprise a reusable material of construction.

It is another objective of the present invention to provide an extremity medical device waterproof cover that may comprise an antimicrobial layer.

It is another objective of the present invention to provide an extremity medical device waterproof cover that may comprise an antimicrobial material of construction.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 is a ventral aspect view of a first embodiment of an extremity medical device waterproof cover, as contemplated by the present disclosure;

FIG. 2 is a dorsal aspect view of a first embodiment of an extremity medical device waterproof cover, as contemplated by the present disclosure;

FIG. 3 is a ventral aspect view of a hand cover of an extremity medical device waterproof cover, as contemplated by the present disclosure;

FIG. 4 is a ventral aspect view of a hand cover of an extremity medical device waterproof cover, as contemplated by the present disclosure;

FIG. 5 is a ventral aspect view of a first embodiment of an extremity medical device waterproof cover shown on an exemplary medical device, as contemplated by the present disclosure;

FIG. 6 is a lateral aspect view of a second embodiment of an extremity medical device waterproof cover, as contemplated by the present disclosure;

FIG. 7 is a medial aspect view of a second embodiment of an extremity medical device waterproof cover, as contemplated by the present disclosure;

FIG. 8 is a caudal aspect view of a foot cover of an extremity medical device waterproof cover, as contemplated by the present disclosure;

FIG. 9 is a caudal aspect view of a foot cover of an extremity medical device waterproof cover, as contemplated by the present disclosure;

FIG. 10 is a caudal aspect view of a foot cover of an extremity medical device waterproof cover, as contemplated by the present disclosure; and

FIG. 11 is a lateral aspect view of a second embodiment of an extremity medical device waterproof cover shown on an exemplary medical device, as contemplated by the present disclosure.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, or in reference to human anatomy in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

The extremity medical device waterproof cover disclosed herein may comprise a waterproof extremity cover combined with digit covers and a plurality of traction pads to provide a user with a waterproof environment for their medical device and additional grip for their hands or feet. The extremity medical device waterproof cover may comprise any appropriate flexible, resilient, and waterproof material and may attach securely to a user's arm or leg by an appropriate cuff.

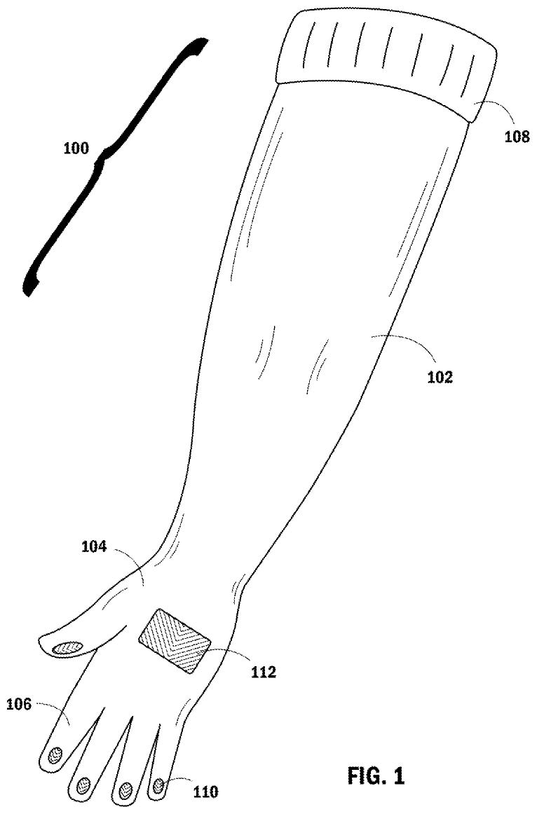

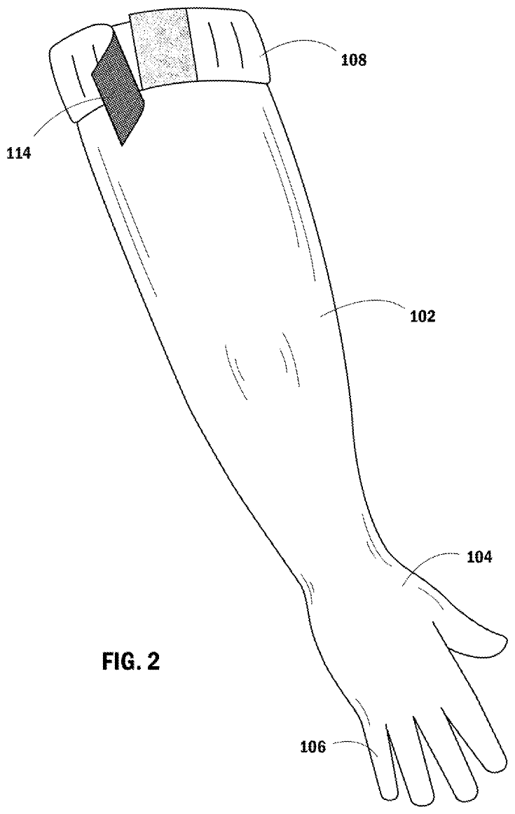

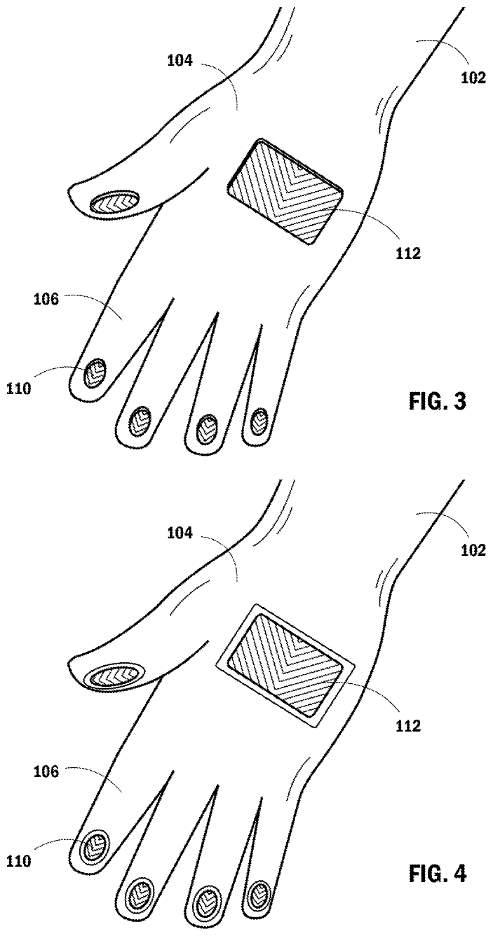

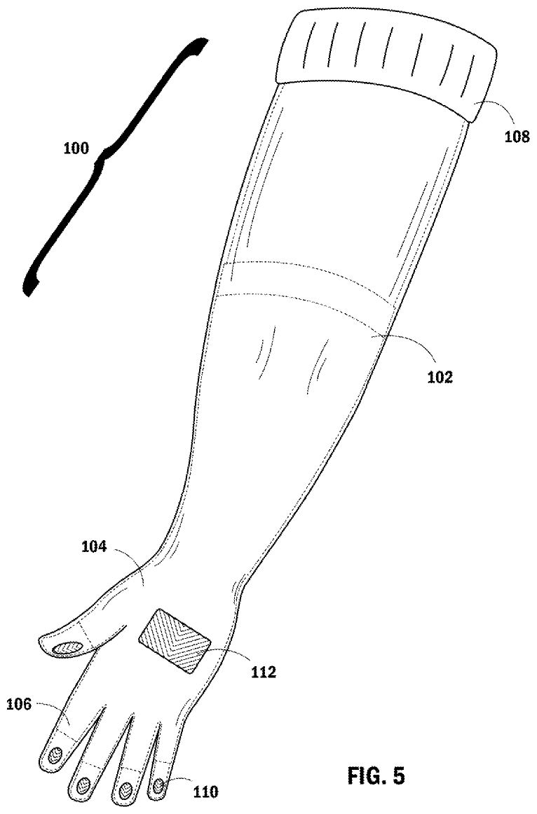

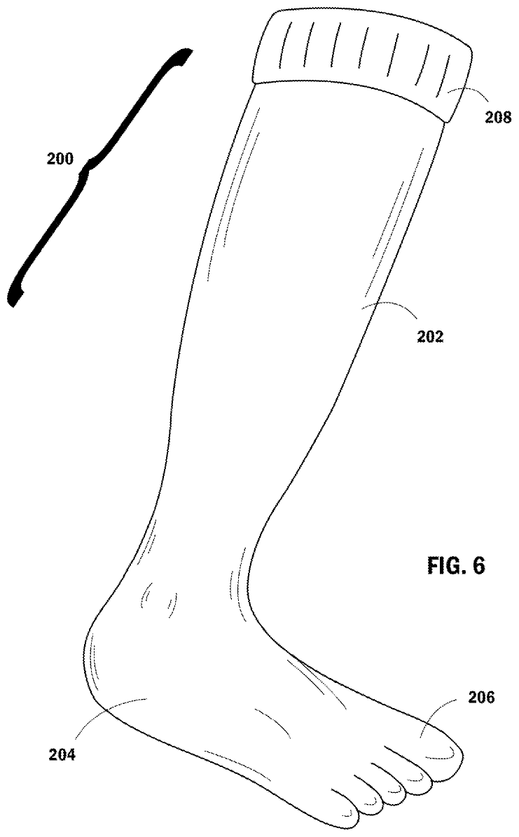

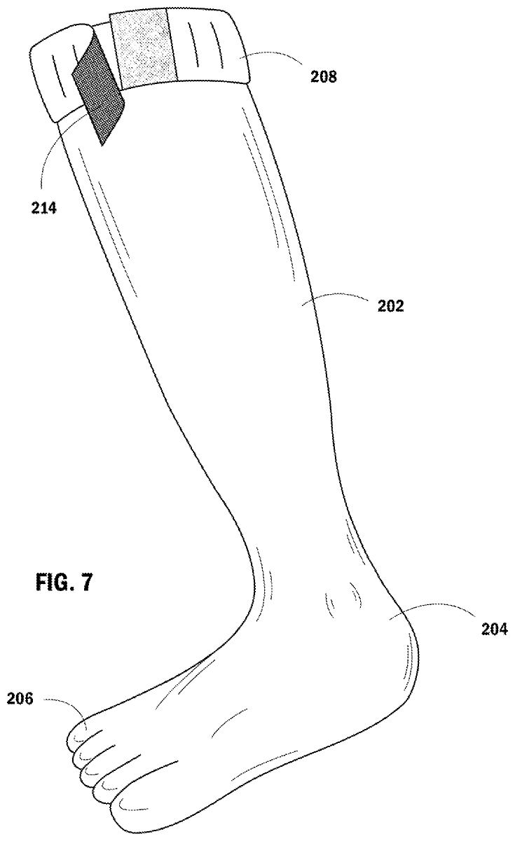

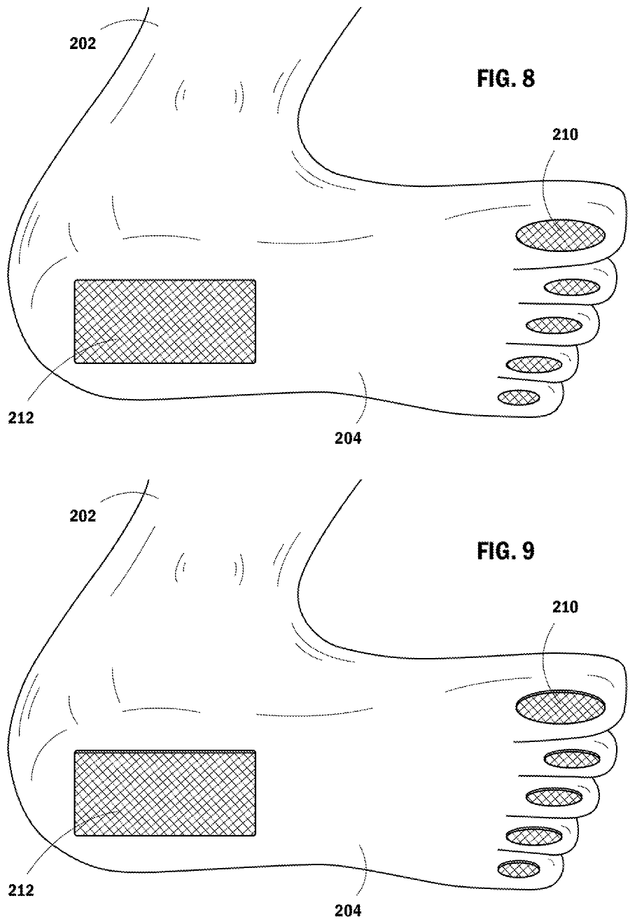

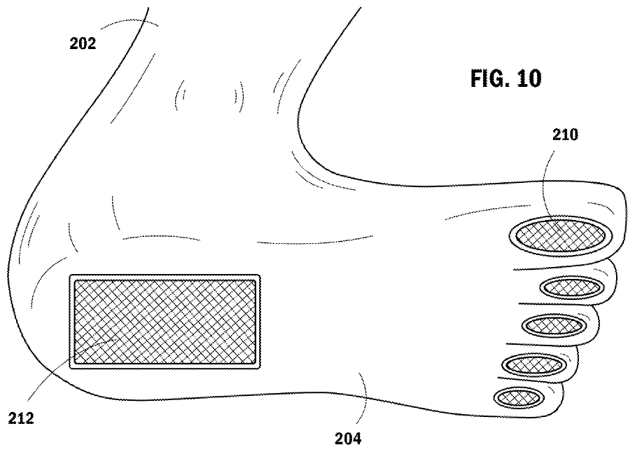

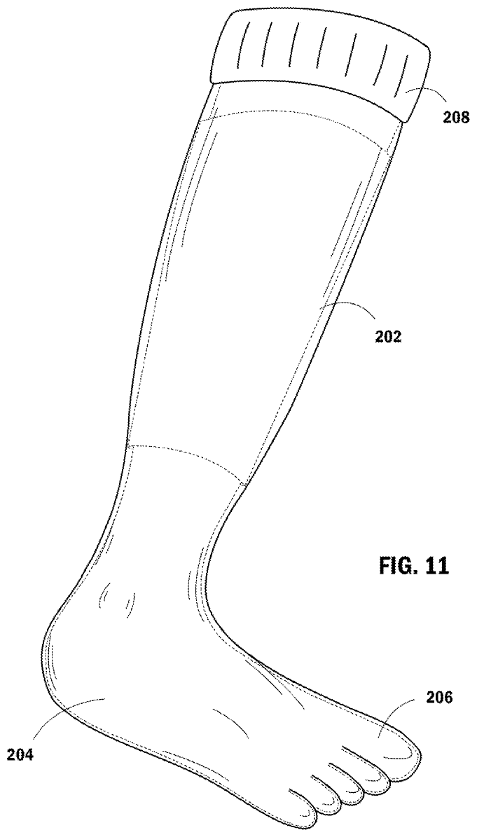

The illustrations of FIGS. 1-11 illustrate an extremity medical device waterproof cover, as contemplated by the present disclosure. A first embodiment of the extremity medical device waterproof cover, which may be an upper extremity (arm) embodiment, may comprise, generally, an arm main body 100, an arm cuff 108, a plurality of finger pads 110, a palm pad 112, and an arm closure mechanism 114.

The arm main body 100 may further comprise an arm sleeve 102, a hand cover 104, and a plurality of finger covers 106 so that the arm main body 100 may cover a user's upper extremity from fingertip to some point along the arm proximal to the user's wrist. Depending on the desired characteristics for a particular embodiment the arm main body 100 may extend to a mid-point of the lower arm, a point just proximal to the elbow, a point just distal to the shoulder, or any other appropriate point.

The arm sleeve 102 of the arm main body 100 may comprise a generally tubular sleeve having a proximal end and a distal end, designed to slip over the arm, and any medical devices installed thereon, of a user. The hand cover 104 may comprise a generally glove-shaped sleeve having a proximal end and a distal end, designed to slip over the hand, and any medical devices installed thereon, of a user. The plurality of finger covers 106 may comprise generally finger-shaped sleeves each having a proximal end and a distal end, designed to slip one each over a user's fingers.

The proximal end of the hand cover 104 may be attached to the distal end of the arm sleeve 102. The proximal ends of the plurality of finger covers 106 may be attached one each to the distal end of the hand cover 104 so as to form a “glove” shape compatible with human anatomy. The attachments between the arm sleeve 102, hand cover 104, and plurality of finger covers 106 may be by any appropriate mechanical means such as, for example, stitching, welding, melting, gluing, adhering, or other similar means. In some embodiments the arm sleeve 102, hand cover 104, and plurality of finger covers 106 may be formed from a single, continuous sheet of material.

The arm cuff 108 may comprise any appropriate mechanism for creating a watertight seal around the arm of a user such as, for example, a waterproof elastic band, a ring and seal combination, a drawstring, a tie string, a tensioning band integrated into the proximal end of the arm sleeve 102, or other similar means. In some embodiments the arm cuff 108 may further comprise a design that provides a watertight seal but is also comfortable for the user so as to prevent chafing or the cutting off of circulation to the arm or the hand.

The arm cuff 108 may further comprise an arm closure mechanism 114, which may comprise any appropriate mechanism for creating a reversible closure that allows for circumferential adjustments of the arm cuff 108 such as, for example, a hook and loop connection, a button snap connection, a strap slide buckle connection, or other similar means.

The plurality of finger pads 110 may comprise any pads appropriate for providing additional tractive effort for the fingers of a user such as, for example, thickened areas of material or attached secondary material on the distal dorsal aspect of each finger cover 106. In an embodiment wherein the plurality of finger pads 110 comprise a thickened area of material they may be integrated into the plurality of finger covers 106 such that they are formed from a single continuous sheet of material. In an embodiment wherein the plurality of finger pads 110 comprise an attached secondary material they may be attached to the plurality of finger covers 106 by any appropriate means such as, for example, stitching, welding, melting, gluing, adhering, or other similar means. The plurality of finger pads 110 may further comprise a textured surface pattern such as, for example, a dimpled, dotted, cross-hatched, or chevroned pattern to provide additional gripping power to the user.

The palm pad 112 may comprise any pad appropriate for providing additional tractive effort for the palm of a user such as, for example, a thickened area of material or attached secondary material on the dorsal aspect of the hand cover 104. In an embodiment wherein the palm pad 112 comprises a thickened area of material it may be integrated into the hand cover 104 such that they are formed from a single continuous sheet of material. In an embodiment wherein the palm pad 112 comprises an attached secondary material it may be attached to the hand cover 104 by any appropriate means such as, for example, stitching, welding, melting, gluing, adhering, or other similar means. The palm pad 112 may further comprise a textured surface pattern such as, for example, a dimpled, dotted, cross-hatched, or chevroned pattern to provide additional gripping power to the user.

A second embodiment of the extremity medical device waterproof cover, which may be a lower extremity (leg) embodiment, may comprise, generally, a leg main body 200, a leg cuff 208, a plurality of toe pads 210, a heel pad 212, and a leg closure mechanism 214.

The leg main body 200 may further comprise a leg sleeve 202, a foot cover 204, and a plurality of toe covers 206 so that the leg main body 200 may cover a user's lower extremity from the tips of the toes to some point along the leg proximal to the user's ankle. Depending on the desired characteristics for a particular embodiment the leg main body 200 may extend to a mid-point of the lower leg, a point just proximal to the knee, a point just distal to the hip, or any other appropriate point.

The leg sleeve 202 of the leg main body 200 may comprise a generally tubular sleeve having a proximal end and a distal end, designed to slip over the leg, and any medical devices installed thereon, of a user. The foot cover 204 may comprise a generally boot-shaped sleeve having a proximal end and a distal end, designed to slip over the foot, and any medical devices installed thereon, of a user. The plurality of toe covers 206 may comprise generally toe-shaped sleeves each having a proximal end and a distal end, designed to slip one each over a user's toes.

The proximal end of the foot cover 204 may be attached to the distal end of the leg sleeve 202. The proximal ends of the plurality of toe covers 206 may be attached one each to the distal end of the foot cover 204 so as to form a “boot” shape compatible with human anatomy. The attachments between the leg sleeve 202, foot cover 204, and plurality of toe covers 206 may be by any appropriate mechanical means such as, for example, stitching, welding, melting, gluing, adhering, or other similar means. In some embodiments the leg sleeve 202, foot cover 204, and plurality of toe covers 206 may be formed from a single, continuous sheet of material.

The leg cuff 208 may comprise any appropriate mechanism for creating a watertight seal around the leg of a user such as, for example, a waterproof elastic band, a ring and seal combination, a drawstring, a tie string, a tensioning band integrated into the proximal end of the leg sleeve 202, or other similar means. In some embodiments the leg cuff 208 may further comprise a design that provides a watertight seal but is also comfortable for the user so as to prevent chafing or the cutting off of circulation to the leg or the foot.

The leg cuff 208 may further comprise a leg closure mechanism 214, which may comprise any appropriate mechanism for creating a reversible closure that allows for circumferential adjustments of the leg cuff 208 such as, for example, a hook and loop connection, a button snap connection, a strap slide buckle connection, or other similar means.

The plurality of toe pads 210 may comprise any pads appropriate for providing additional tractive effort for the toes of a user such as, for example, thickened areas of material or attached secondary material on the distal caudal aspect of each toe cover 206. In an embodiment wherein the plurality of toe pads 210 comprise a thickened area of material they may be integrated into the plurality of toe covers 206 such that they are formed from a single continuous sheet of material. In an embodiment wherein the plurality of toe pads 210 comprise an attached secondary material they may be attached to the plurality of toe covers 206 by any appropriate means such as, for example, stitching, welding, melting, gluing, adhering, or other similar means. The plurality of toe pads 210 may further comprise a textured surface pattern such as, for example, a dimpled, dotted, cross-hatched, or chevroned pattern to provide additional gripping power to the user.

The heel pad 212 may comprise any pad appropriate for providing additional tractive effort for the heel of a user such as, for example, a thickened area of material or attached secondary material on the caudal aspect of the foot cover 204. In an embodiment wherein the heel pad 212 comprises a thickened area of material it may be integrated into the foot cover 204 such that they are formed from a single continuous sheet of material. In an embodiment wherein the heel pad 212 comprises an attached secondary material it may be attached to the foot cover 204 by any appropriate means such as, for example, stitching, welding, melting, gluing, adhering, or other similar means. The heel pad 212 may further comprise a textured surface pattern such as, for example, a dimpled, dotted, cross-hatched, or chevroned pattern to provide additional gripping power to the user.

To begin using the upper extremity (arm) embodiment of the device a user may first place their hand and arm into the arm cuff 108 and slip the device proximally over their arm until their fingers are secured within one each of the finger covers 106. The user may then tighten and close the arm closure mechanism 114 to create a watertight interior compartment within which is contained the user's hand, a portion of their arm, and any medical devices installed thereon. By correctly placing their fingers within one each of the finger covers 106 the user will have the benefit of one each of the finger pads 110 being placed dorsally to their fingertips, and the palm pad 112 being placed dorsally to their palm. The user may then grip objects as desired while keeping their medical device clean and dry. To remove the device the user may reverse the closure of the arm closure mechanism 114 and slip the device off of their arm.

To begin using the lower extremity (leg) embodiment of the device a user may first place their foot and leg into the leg cuff 208 and slip the device proximally over their leg until their toes are secured within one each of the toe covers 206. The user may then tighten and close the leg closure mechanism 214 to create a watertight interior compartment within which is contained the user's foot, a portion of their leg, and any medical devices installed thereon. By correctly placing their toes within one each of the toe covers 206 the user will have the benefit of one each of the toe pads 210 being placed caudally to their toes, and the heel pad 212 being placed caudally to their heel. The user may then grip objects as desired while keeping their medical device clean and dry. To remove the device the user may reverse the closure of the leg closure mechanism 214 and slip the device off of their leg.

The extremity medical device waterproof cover may be substantially constructed of any suitable material or combination of materials, but typically is constructed of a resilient material or combination of materials such that the device is easily manufactured, insulating, and reusable. As an example, and without limiting the scope of the present invention, various exemplary embodiments of the extremity medical device waterproof cover may be substantially constructed of one or more materials of plastic, acrylic, polyethylene, polypropylene, polycarbonate, fabric, denim, steel, aluminum, brass, fiberglass, carbon fiber, or combinations thereof. In some embodiments the various components of the device may be coated, lined, or otherwise insulated to prevent contamination of the device.

In one embodiment the extremity medical device waterproof cover may comprise a resilient material of construction that either comprises a material having antimicrobial properties or comprises a layering of antimicrobial material or coating. Antimicrobial properties comprise the characteristic of being antibacterial, biocidal, microbicidal, anti-fungal, anti-viral, or other similar characteristics, and the oligodynamic effect, which is possessed by copper, brass, silver, gold, and several other metals and alloys, is one such characteristic. Copper and its alloys, in particular, have exceptional self-sanitizing effects. Silver also has this effect, and is less toxic to users than copper. Some materials, such as silver in its metallic form, may require the presence of moisture to activate the antimicrobial properties.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

I claim:

1. An extremity medical device waterproof cover, comprising:
a main body;
a cuff;
a closure mechanism;
a plurality of digit pads; and
a secondary pad;
wherein said main body further comprises a tubular sleeve having a proximal end and a distal end, a cover having a proximal end and a distal end, and a plurality of digit covers each having a proximal end and a distal end;
wherein said proximal end of one each of said plurality of digit covers is attached to said distal end of said cover;
wherein said proximal end of said cover is attached to said distal end of said tubular sleeve;
wherein said proximal end of said tubular sleeve is attached to said cuff;
wherein said closure mechanism is integrated into said cuff;
wherein one each of said plurality of digits pads is attached to one each of said plurality of digit covers; and
wherein said secondary pad is attached to said cover.

2. An extremity medical device waterproof cover, comprising:
an arm main body;
an arm cuff;
an arm closure mechanism;
a plurality of finger pads; and
a palm pad;
wherein said arm main body further comprises an arm sleeve having a proximal end and a distal end, a hand cover having a proximal end and a distal end, and a plurality of finger covers each having a proximal end and a distal end;
wherein said hand cover comprises a glove shape;
wherein said proximal end of one each of said plurality of finger covers is attached to said distal end of said hand cover;
wherein said proximal end of said hand cover is attached to said distal end of said arm sleeve;
wherein said proximal end of said arm sleeve is attached to said arm cuff;
wherein said arm closure mechanism is integrated into said arm cuff;
wherein one each of said plurality of finger pads is attached to one each of said plurality of finger covers; and
wherein said palm pad is attached to said hand cover.

3. The waterproof cover of claim 2,
wherein one each of said plurality of finger pads is attached to a dorsal side of one each of said plurality of finger covers; and
wherein said palm pad is attached to a dorsal side of said hand cover.

4. The waterproof cover of claim 2,
wherein said arm cuff comprises an elastic band.

5. The waterproof cover of claim 2,
wherein said arm closure mechanism comprises a hook and loop fastener.

6. An extremity medical device waterproof cover, comprising:
a leg main body;
a leg cuff;
a leg closure mechanism;
a plurality of toe pads; and
a heel pad;
wherein said leg main body further comprises a leg sleeve having a proximal end and a distal end, a foot cover having a proximal end and a distal end, and a plurality of toe covers each having a proximal end and a distal end;
wherein said foot cover comprises a boot shape;
wherein said proximal end of one each of said plurality of toe covers is attached to said distal end of said foot cover;
wherein said proximal end of said foot cover is attached to said distal end of said leg sleeve;
wherein said proximal end of said leg sleeve is attached to said leg cuff;
wherein said leg closure mechanism is integrated into said leg cuff;
wherein one each of said plurality of toe pads is attached to one each of said plurality of toe covers; and
wherein said heel pad is attached to said foot cover.

7. The waterproof cover of claim 6,
wherein one each of said plurality of toe pads is attached to a caudal side of one each of said plurality of toe covers; and
wherein said heel pad is attached to a caudal side of said foot cover.

8. The waterproof cover of claim 6,
wherein said leg cuff comprises an elastic band.

9. The waterproof cover of claim 6,
wherein said leg closure mechanism comprises a hook and loop fastener.
